(19) United States
(12) Patent Application Publication
(10) Pub. No.: US 2013/0137952 A1
(43) Pub. Date: May 30, 2013

(54) METHOD AND SYSTEM TO DISPLAY ANALYTE SENSOR DATA

(71) Applicants: Thomas McCann, Pottstown, PA (US); Eric Keller, Norristown, PA (US); Thomas Fuhs, Phoenixville, PA (US); David Walton, Newtown, PA (US); John Porter, Ridley Park, PA (US); Brian McLaughlin, Media, PA (US)

(72) Inventors: Thomas McCann, Pottstown, PA (US); Eric Keller, Norristown, PA (US); Thomas Fuhs, Phoenixville, PA (US); David Walton, Newtown, PA (US); John Porter, Ridley Park, PA (US); Brian McLaughlin, Media, PA (US)

(73) Assignee: Animas Corporation, West Chester, PA (US)

(21) Appl. No.: 13/637,277

(22) PCT Filed: Mar. 23, 2011
(86) PCT No.: PCT/US11/29618

§ 371 Date: Dec. 17, 2012

Related U.S. Application Data
(60) Provisional application No. 61/319,736, filed on Mar. 31, 2010.

(51) Int. Cl. A61B 5/00; A61M 5/172; A61B 5/145

(52) U.S. Cl. CPC A61B 5/743; A61B 5/14532; A61B 5/4839; A61B 5/14503; A61B 5/0004; A61M 5/1723
USPC 600/365; 600/309; 604/66

(57) ABSTRACT

Described and illustrated herein are an exemplary methods and system to display analyte sensor data. Such method may be achieved by the medical device receiving data from the analyte sensor for a period of time and displaying a target zone and a graphical representation of the data in which one or more portions of the graphical representation comprises a specific indicia of clinical risk.

This application claims the benefits of priority under 35 USC§119 and/or §120 from prior filed U.S. Provisional Application Ser. No. 61/319,736 filed on Mar. 31, 2010, which application is incorporated by reference in its entirety into this application.

### BACKGROUND

Analyte monitoring systems including continuous and discrete monitoring systems generally include a small, lightweight battery powered and microprocessor controlled system which is configured to detect signals proportional to the corresponding measured analyte levels and RF signals to transmit the collected data. One aspect of certain analyte monitoring systems includes a transcutaneous or subcutaneous analyte sensor configuration which is, for example, partially mounted on the skin of a subject whose analyte level is to be monitored. The sensor cell may use a two or three-electrode (work, reference and counter electrodes) configuration driven by a controlled potential (potentiostat) analog circuit connected through a contact system.

The analyte sensor may be configured so that a portion thereof is placed proximate the skin of the patient so as to detect the analyte levels of the patient and another portion or segment of the analyte sensor is in communication with the transmitter unit. The transmitter unit is configured to transmit the analyte levels detected by the sensor over a wireless communication link such as, for example, an RF (radio frequency) communication link to a receiver/monitor unit such as a remote controller, an infusion pump or a blood glucose meter. The receiver/monitor unit performs data analysis on the received analyte levels to generate information pertaining to the monitored analyte levels. The receiver/monitor unit may also display the data received analyte levels.

### SUMMARY OF THE DISCLOSURE

Applicants have recognized a need for a system and method that can be used to display analyte sensor data that are easy to understand with lower risk of confusion, especially when the user is in a clinical risk zone.

In view of the foregoing and in accordance with one aspect, there is provided a method of displaying analyte sensor data on a medical device such as an infusion pump or analyte meter. The medical device includes a display, an RF transmitter/receiver and a microprocessor to process the data received from the analyte sensor. The method of displaying data from an analyte sensor may be by determining with an analyte sensor that transforms signals of analyte concentrations in physiological fluid of the user over a period of time into determined analyte concentration data; and displaying a target zone of upper and lower analyte concentration thresholds, a first indicia representative of the determined analyte concentration data in the target zone, and a second indicia representative of the determined analyte concentration data outside of the target zone. In this method, the displaying further comprises displaying a third indicia for at least one analyte concentration of the determined analyte concentration data below the target zone and the target zone comprises a threshold value indicative of clinical risks arising from diabetes. The aforementioned method may include the following features: each of the first, second and third indices comprise respective symbols; each of the indices further comprises a color; each of the indices comprises an icon; and the analyte sensor comprises a continuous glucose sensor. In such method, the displaying comprises displaying a trend indicative symbol to represent a trend of determined analyte concentration data over a predetermined time frame.

In yet another embodiment, a system for diabetes management is provided that includes an analyte sensor, infusion pump and a display. The analyte sensor is configured to transform analyte measurements conducted by the analyte sensor into signals representative of the analyte measurements over time. The infusion pump includes a portable microprocessor device configured to receive data from the analyte sensor. The display exhibits a target zone of upper and lower analyte concentration thresholds, a first indicia representative of the determined analyte concentration data in the target zone, and a second indicia representative of the determined analyte concentration data outside of the target zone. The aforementioned system may include the following features: each of the indices comprises a color; each of the indices comprises a symbol; each of the indices comprises an icon; and the display comprises an OLED disposed in a housing of the infusion pump. In such system, the display comprises a display configured to exhibit a trend indicative symbol to represent a trend of the determined analyte concentration over a predetermined time frame.

In a further embodiment, a system for processing and displaying data from an analyte sensor is provided. The system includes an analyte sensor and handheld portable microprocessor device. The analyte sensor transforms analyte measurements conducted by the analyte sensor into signals representative of the analyte measurements. The handheld portable microprocessor device is configured to receive signals from the analyte sensor. The handheld portable device includes a screen that displays a target zone of upper and lower analyte concentration thresholds, a first indicia representative of the determined analyte concentration data in the target zone, and a second indicia representative of the determined analyte concentration data outside of the target zone. The aforementioned system may include the following features: each of the first and second indicia comprises a color; each of the first and second indicia comprises a symbol; each of the first and second indicia comprises an icon; and the analyte sensor comprises a continuous glucose sensor.

In yet a further embodiment, a method of monitoring analyte concentrations of a user over time is provided. The method can be achieved by: receiving data from a sensor that transforms signals representative of analyte concentrations in a physiological fluid of the user over a period of time; and displaying a target zone of upper and lower analyte concentration thresholds, a first indicia representative of the data representative of analyte concentrations in the target zone, and a second indicia representative of the received data representative of analyte concentrations outside of the target zone. The aforementioned method may include the following features: the displaying further comprises displaying a third indicia for at least one analyte concentration below the target zone; the target zone comprises a threshold value indicative of clinical risks arising from diabetes; each of the first, second and third indices comprises respective symbols; each of the indices further comprises a color; or each of the indices comprises an icon.

These and other embodiments, features and advantages will become apparent to those skilled in the art when taken with reference to the following more detailed description of the invention in conjunction with the accompanying drawings that are first briefly described.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements), of which:

FIG. 1A illustrates an exemplary embodiment of a top view of a system for continuously measuring an analyte;

FIG. 1B illustrates an exemplary analyte sensor for the system of FIG. 1A.

FIG. 2 illustrates another exemplary embodiment of a top view of a system for continuously measuring an analyte;

FIG. 3 illustrates an exemplary embodiment of a flow chart of a method of displaying data from an analyte sensor; and

FIG. 4 illustrates an exemplary embodiment of a display displaying data from an analyte sensor.

### MODES OF CARRYING OUT THE INVENTION

The following detailed description should be read with reference to the drawings, in which like elements in different drawings are identically numbered. The drawings, which are not necessarily to scale, depict selected exemplary embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient”, “host”, “user”, and “subject” refer to any human or animal subject and are not intended to limit the systems or methods to human use, although use of the subject invention in a human patient represents a preferred embodiment.

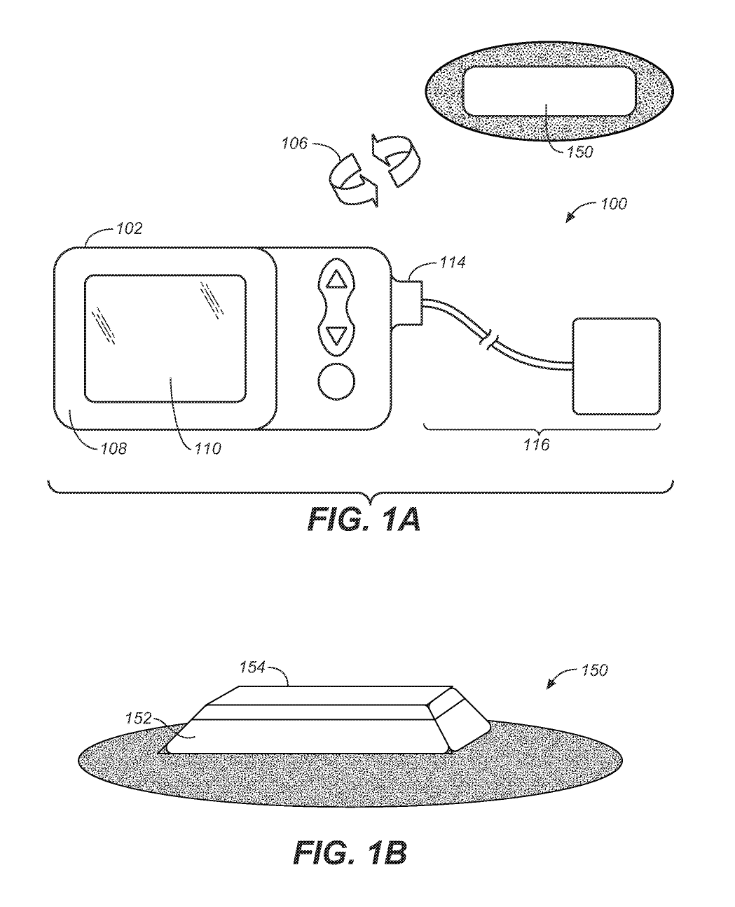

FIG. 1A illustrates a system 100 for continuously measuring an analyte in which the system 100 includes an infusion pump 102 and a sensor 150. The infusion pump 102 is configured to transmit and receive data to and from the sensor 150 by, for example, radio frequency communication 106. The sensor 150 is inserted subcutaneously and may measure an analyte such as glucose in the physiological fluid (e.g., blood, interstitial, saliva, sweat and the like) of the user. The measurements or assays of the analyte are transformed into signal data that are transmitted to a remote device such as, for example, an infusion pump, analyte meter, or a portable handheld microprocessor device (e.g., a mobile phone including a Smartphone). The infusion pump 102 includes a housing 108, a display 110 for providing operational information to the user, a plurality of navigational buttons 112 for the user to input information, a battery in a compartment (not shown) for providing power to the infusion pump 102, a drug delivery mechanism (e.g., a pump and drive mechanism; not shown) for forcing a drug such as insulin from a cartridge in a chamber through a side port 114 connected to an infusion set 116 and into the body of the user. The infusion pump 102 further includes electronic circuitry within housing 108 such as a memory and a microprocessor (not shown). Details of the electronic circuitry of such pump are shown and described in US Patent Application Publication No. US 2009-0099505, which is hereby incorporated by reference in its entirety herein. The display 110 may output a graphical representation of the analyte concentrations measured by the sensor 150. The display 110 may be used to show a user interface for prompting a user on how to operate the infusion pump102. The plurality of navigational buttons 112 allow a user to operate infusion pump 102 by navigating through the user interface software. The display 110 may optionally include a backlight. Various examples of infusion pumps may be utilized. For example, an infusion pump from Animas Corporation (Animas Model 2020 or Animas Ping System) may be utilized.

Referring to FIG. 1B, a suitable analyte sensor 150 may assays or measures analyte concentrations in physiological fluids (e.g., blood, saliva or interstitial fluids) via sensor probes (not shown) and transmit such data via a transmitter 154 to a receiver (e.g., pump 110, meter 220, phones 240 or 260). Details of the sensor 150 are shown and described in U.S. Pat. No. 6,931,327, which is hereby incorporated by reference in its entirety herein. A commercial embodiment of the sensor 150 utilized in a monitoring system is available from DexCom™ Inc., as model Seven Plus system. Another commercially available infusion pump and CGM system may also be utilized such as, for example, the MiniMed™ Paradigm Revel System. Yet another continuous sensor that may be utilized is described in U.S. Pat. No. 6,144,869, which is incorporated by reference herein this application.

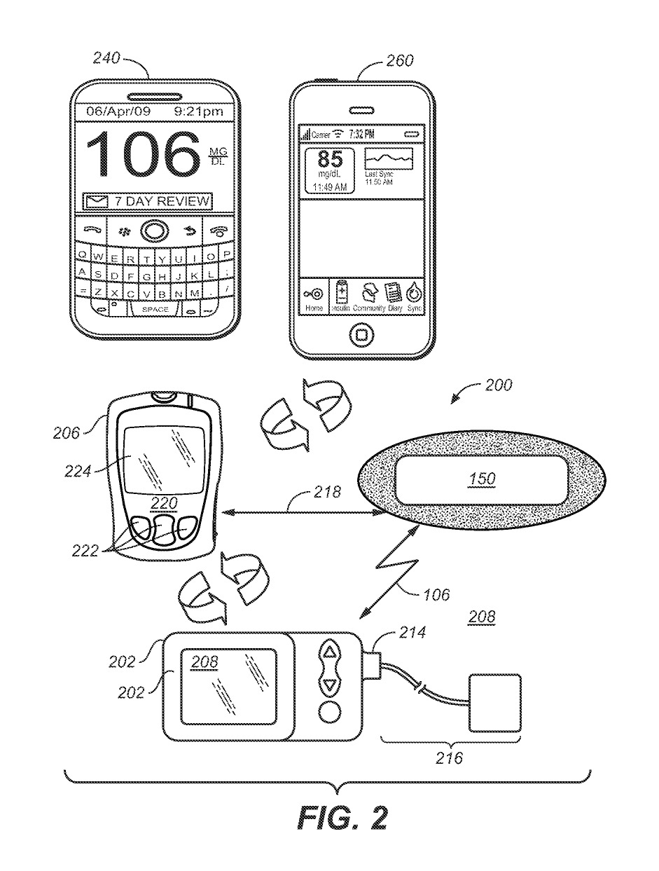

FIG. 2 illustrates another system 200 for continuously measuring an analyte in which the system 200 includes a pump 202 (which may not have a display), a sensor 150, a meter 206, and one or more mobile computing devices 240 and 260. The pump 202 is operated manually or semi-automatically and includes a housing 208 and a side port 214 connected to an infusion set 216 through which a drug is delivered from a cartridge (not shown) into the body of the user. The meter 206 is configured to transmit and receive data to and from the sensor 150 by, for example, radio frequency communication 218. The sensor has probes that may be inserted subcutaneously and may measure an analyte such as glucose. The meter 206 includes a housing 220, a plurality of navigational buttons 222 for the user to input information, and a display 224 for providing operational information to the user and for outputting a graphical representation of the data from the sensor 150. The plurality of navigational buttons 222 allow a user to operate the meter 206 by navigating through the user interface software. The display 224 may optionally include a backlight. The meter 206 further includes electronic circuitry such as a memory and a microprocessor (not shown) for processing data received from the sensor 150. The pump 202 may be configured to operate without a built-in display screen due the use of the display screen of meter 206 being utilized in place of the display for the pump 202.

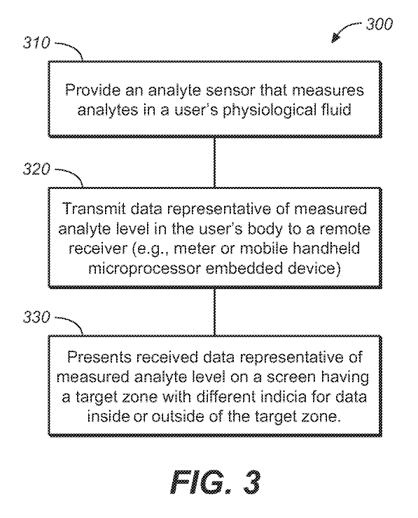

Referring to FIG. 3, a method 300 for diabetes management that uses the aforementioned system embodiments 100 or 200 will now be described. In exemplary step 310, an analyte sensor and a medical device (e.g., an infusion pump or an analyte meter) with a display are provided, as shown in FIG. 1A or FIG. 2. Preferably, the sensor 150 is attached to a patient or user and probes in the sensor perform electrochemical assays to determine the analyte (e.g., glucose or ketone and the like) concentrations or values in the body of the patient. The sensor 150 transforms the signals derived from the electrochemical assay of a user's physiological fluids by the sensor into analyte signals representative of the analyte concentrations in the user's physiological fluid. The analyte signals are then transmitted to a remote receiver (or transceiver) such as the pump, meter, or mobile handheld microprocessor embedded device as data signals. Alternatively, the analyte sensor and the medical device are configured to transmit and receive data wirelessly to and from each other.

In exemplary step 320, the medical device receives data from the analyte sensor 150 for a period of time. The received analyte signals are transformed into pixels on a display to represent a tangible quantity of the analyte (in this case, glucose) concentrations over time in the user's body.

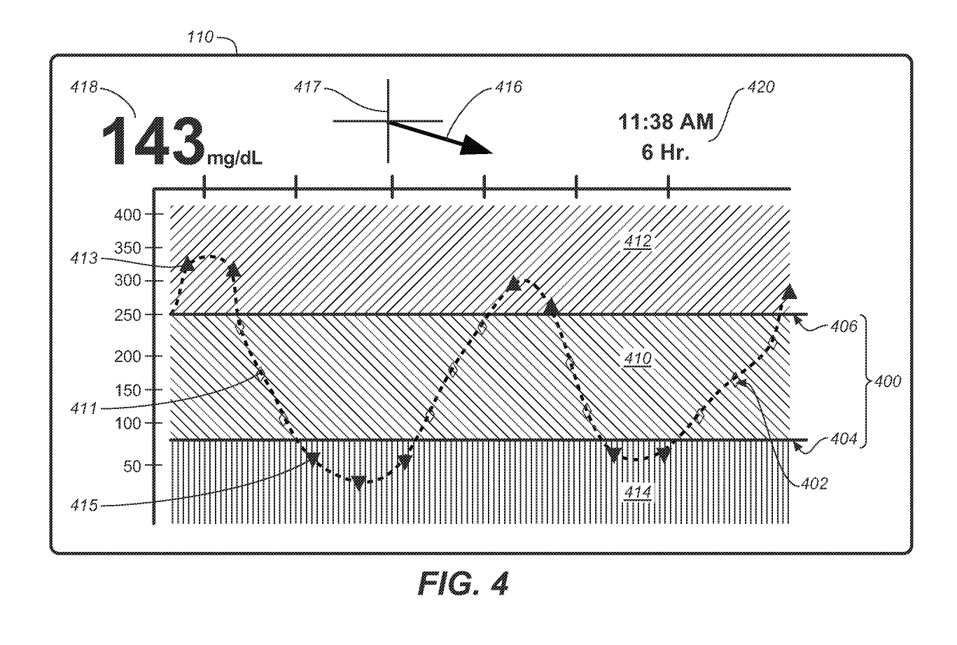

In exemplary step 330, the medical device displays a target zone 400 and a graphical representation 402 of the data in which one or more portions of the graphical representation include a specific indicia of clinical risk. An exemplary display 110 for the collected analyte data is illustrated in FIG. 4 in which the glucose concentration is plotted as a function of time. The target zone 400 is represented by a lower threshold 404 and an upper threshold 406 (shown as horizontal lines) above or below which there is believed to be clinical risk. Below the lower threshold 404, a diabetic may be at risk for hypoglycemia and above the upper threshold 406, a diabetic may be at risk for hyperglycemia. In the embodiment shown in FIG. 4, the lower threshold 404 is set at about 50 mg/dL glucose and the upper threshold 406 is set at about 250 mg/dL glucose. The upper and lower thresholds 404 and 406 for clinical risk are typically defined by the health care provider. The specific indicia of clinical risk may be color, symbols or icons. In the embodiment shown in FIG. 4, the system changes the symbols (e.g., from symbol 413 “▾” to 411 “⋄” and to 415 “▴”) when the data is above or below the upper or lower thresholds, respectively, of clinical risk. In another embodiment, the graph changes color (e.g., in shaded area 410 having a color such as green to shaded area 412 having a color such as amber) or to red shaded area 414 when the data is in, above or below the upper or lower threshold, respectively, of clinical risks. In an alternate embodiment, the display can plot other relevant data on the same graph in yet another color. For example, the display would plot episodic blood glucose values in white on the same graph with different color shaded areas. Another embodiment would be to mark bolus deliveries and or basal changes along the (horizontal) time axis with respect to insulin units delivered along the vertical axis. This would give the user visual feedback about the relative effectiveness of their insulin therapy regimen.

By virtue of the use of different indices to indicate different areas of clinical risk, the user is presented with information that are easy to understand with lower risk of confusion. For example, the use of the indicia 413 as the symbol ▴ above the threshold line 406 allows the user to quickly assess that in the last 6 hours, there have been 5 instances of potential hyperglycemia in the amber zone 412. Similarly, the use of the indicia 415 as the symbol ▾ allows the user to quickly assess that in the last 6 hours, there have been 5 instances of potential hypoglycemia in the red zone 414. An icon such as that representing an ambulance can be utilized in place of the symbol 415 to signify that these represents a potential emergency, and an icon representing a doctor can be utilize in place of the indicia 413 to signify that the user would need to discuss with a healthcare provider regarding these higher analyte values.

As illustrated in the embodiment in FIG. 4, the display may also show the current level of analyte at 418, the direction the data is trending by, for example, an arrow 416 in relation to a baseline 417, the time and other information 420 as needed. The display can be any type of display including, for example, a Liquid-Crystal-Display, or preferably an Organic-Light-Emitting-Diode display.

In conclusion, the system and methods described and illustrated herein can be used to display data from an analyte sensor such that the graphical representation gives specific indicia of clinical risk. Although the specific embodiments have been described in relation to a continuous analyte sensor, other analyte sensors may also be utilized as long as data signals representative of analyte quantity in a user's body are provided to the handheld device.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A method of monitoring analyte concentrations of a user over time, the method comprising:
determining with an analyte sensor that transforms signals of analyte concentrations in physiological fluid of the user over a period of time into determined analyte concentration data; and
displaying a target zone of upper and lower analyte concentration thresholds, a first indicia representative of the determined analyte concentration data in the target zone, and a second indicia representative of the determined analyte concentration data outside of the target zone.

2. The method of claim 1, in which the displaying further comprises displaying a third indicia for at least one analyte concentration of the determined analyte concentration data below the target zone.

3. The method of claim 1, in which the target zone comprises a threshold value indicative of clinical risks arising from diabetes.

4. The method of claim 2, in which each of the first, second and third indices comprises respective symbols.

5. The method of claim 2, in which each of the indices further comprises a color.

6. The method of claim 1, in which each of the indices comprises an icon.

7. The method of claim 1, in which the analyte sensor comprises a continuous glucose sensor.

8. The method of claim 1, in which the displaying comprises displaying a trend indicative symbol to represent a trend of determined analyte concentration data over a predetermined time frame.

9. A system for diabetes management, the system comprising:
an analyte sensor configured to transmit data transformed from a user's signals representing analyte concentrations over time;
an infusion pump including a microprocessor configured to receive data from the analyte sensor; and
a display connected to the microprocessor that exhibits a target zone of upper and lower analyte concentration thresholds, a first indicia representative of the determined analyte concentration data in the target zone, and a second indicia representative of determined analyte concentration data outside of the target zone.

10. The system of claim 9, in which each of the indices comprises a color.

11. The system of claim 9, in which each of the indices comprises a symbol.

12. The system of claim 9, in which each of the indices comprises an icon.

13. The system of claim 9, in which the display comprises an OLED disposed in a housing of the infusion pump.

14. The system of claim 9, in which the display comprises a display configured to exhibit a trend indicative symbol to represent a trend of the determined analyte concentration over a predetermined time frame.

15. A system for processing and displaying data from an analyte sensor, the system comprising:
an analyte sensor that transforms analyte measurements conducted by the analyte sensor into signals representative of the analyte measurements;
a handheld portable microprocessor device configured to receive signals from the analyte sensor, the handheld portable device including a screen that displays a target zone of upper and lower analyte concentration thresholds, a first indicia representative of the determined analyte concentration data in the target zone, and a second indicia representative of the determined analyte concentration data outside of the target zone.

16. The system of claim 15, in which each of the first and second indicia comprises a color.

17. The system of claim 15, in which each of the first and second indicia comprises a symbol.

18. The system of claim 15, in which each of the first and second indicia comprises an icon.

19. The system of claim 15, in which the analyte sensor comprises a continuous glucose sensor.

20. A method of monitoring analyte concentrations of a user over time, the method comprising:
receiving data from a sensor that transforms signals representative of analyte concentrations in a physiological fluid of the user over a period of time; and
displaying a target zone of upper and lower analyte concentration thresholds, a first indicia representative of the data representative of analyte concentrations in the target zone, and a second indicia representative of the received data representative of analyte concentrations outside of the target zone.

21. The method of claim 20, in which the displaying further comprises displaying a third indicia for at least one analyte concentration below the target zone.

22. The method of claim 20, in which the target zone comprises a threshold value indicative of clinical risks arising from diabetes.

23. The method of claim 21, in which each of the first, second and third indices comprises respective symbols.

24. The method of claim 21, in which each of the indices further comprises a color.

25. The method of claim 20, in which each of the indices comprises an icon.
